(19) United States
(12) Patent Application Publication
(10) Pub. No.: US 2004/0231722 A1
(43) Pub. Date: Nov. 25, 2004

(54) Method for preventing clogging of apparatus for handling (meth) acrylic acid and esters thereof

(76) Inventors: Shuhei Yada, Yokkaichi-shi (JP); Kimikatsu Jinno, Yokkaichi-shi (JP); Yasushi Ogawa, Yokkaichi-shi (JP); Yoshiro Suzuki, Yokkaichi-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/879,228

(22) Filed: Jun. 30, 2004

Related U.S. Application Data
(63) Continuation of application No. PCT/JP03/00063, filed on Jan. 8, 2003.

(30) Foreign Application Priority Data
Jan. 8, 2002 (JP) 2002-1231

(51) Int. Cl. C07C 57/04

(52) U.S. Cl. 137/240

(57) ABSTRACT

In a nozzle or a pipe for connecting a measuring device which monitors a process state of (meth)acrylic acid and esters thereof and a gas-phase part and/or a liquid-phase part of an apparatus for handling (meth)acrylic acid and esters thereof, a gas comprising at least one of an inert gas, oxygen, and a gas having an effect of inhibiting polymerization is blown into the nozzle and/or the pipe connected to the gas-phase part of the apparatus at a flow rate of 0.03 to 1 m/second and a liquid medium is blown into the nozzle and/or the pipe connected to the liquid-phase part of the apparatus at a flow rate of 0.03 to 1 m/second to prevent a clogging of the apparatus for handling (meth)acrylic acid and esters thereof. According to the method, stable and efficient operation of the apparatus for handling (meth)acrylic acid and the like and a decrease of a cost for production or storage of (meth)acrylic acid can be achieved.

### TECHNICAL FIELD

The present invention relates to a method for preventing clogging of an apparatus for handling (meth)acrylic acid and esters thereof and to realize a safe and low-cost operation.

### BACKGROUND ART

It has been well known that (meth)acrylic acid and esters thereof are very easily polymerizable and in a step for producing them and use of the compounds, polymerization occurs frequently, resulting in that the operation of an apparatus is inevitably stopped. For this reason, to prevent extraordinary polymerization, a countermeasure of addition of polymerization inhibitors such as hydroquinone and copper-containing compounds into an apparatus for handling (meth)acrylic acid and esters thereof has been adopted.

However, these polymerization inhibitors have their polymerization inhibition activities only in liquid-phases. Therefore, to allow gas-phase or a liquid formed by liquefaction of the gas-phase to have a polymerization inhibition activity, a gas comprising oxygen (air or a gas whose oxygen concentration is adjusted by mixing an inert gas such as nitrogen, carbon dioxide, or argon) has to be used to make use of the polymerization inhibition activity of oxygen. From this, it follows that usually, a method of operation with blowing the gas comprising oxygen into the apparatus when (meth)acrylic acid and esters thereof are handled is adopted.

An appliance, such as a distillation column, an evaporator, a heat exchanger, and a tank, is used as apparatus that handles (meth)acrylic acid and the like in a manner similar to that in usual chemical plants. To continuously operate the apparatus and achieve safe operation at low cost, it is essential to measure a process state such as pressure, temperature, and liquid level. It is necessary to install a nozzle or the like to the aforementioned appliance and connect a measuring appliance thereto in order to measure the process state. However, in the case of the nozzle or the like, usually continuous renewal of a process fluid occurs less frequently, resulting in a prolonged residence time and insufficient supply of an inhibitor, so that generation of a polymer at this part is frequently observed.

The generation of a polymer at the part concerned not only makes proper measurement of the process state impossible but also makes stable operation impossible since the generated polymer may serve as a nucleus for polymer generation, go with the process liquid to other parts, and generate a polymer at the other parts.

To prevent these problems, a method is adopted in which stagnation of the liquid is prevented by blowing an inert gas or the gas comprising oxygen into the nozzle or the like connected to a gas-phase part, or by blowing a liquid comprising a polymerization inhibitor into the nozzle or the like connected to a liquid-phase part.

However, in the method of blowing an inert gas or a gas comprising oxygen into the nozzle or the like connected to the gas-phase part, the appliance is frequently operated under reduced pressure. Therefore, in this case, an amount of gas discharged from a decompressing apparatus such as a vacuum pump or an ejector increases. In accordance with this, a size of the decompressing apparatus increases, and the problems arise that troubles occur due to polymerization of (meth)acrylic acid and the like in the decompressing apparatus or downstream thereof due to an increase of the amount of (meth)acrylic acid discharged outside the system accordingly.

Further, since the aforementioned gas is blown into the nozzle or the like even when the appliance is used at an atmospheric pressure, a step of treating the gas discharged from the appliance in a scrubber, a combustion furnace or the like becomes necessary, resulting in high cost due to provision of a treating apparatus.

Also for the method of blowing the liquid comprising a polymerization inhibitor into the liquid-phase part, an increase in the amount of the liquid requires an increase in size of a pump that discharges the liquid, resulting in an increased consumption of energy for driving such as electricity, steam, or gas as compared with the case where no liquid is blown in. As a result, the cost for handling (meth)acrylic acid and the like increases.

### DISCLOSURE OF THE INVENTION

The present invention has been achieved in view of the aforementioned problems and it is an object of the present invention to operate an apparatus for handling (meth)acrylic acid and the like stably and efficiently to reduce the cost for producing and storing (meth)acrylic acid.

The inventors of the present invention have found that blowing a specified gas or liquid at a flow rate within a specified range into an apparatus for handling (meth)acrylic acid and esters thereof and a nozzle or a pipe that connects the apparatus to a measuring device for monitoring a process state can suppress the generation of polymerization of (meth)acrylic acid and the like to prevent the nozzle from being clogged with polymerisates or solids, thereby have achieved the present invention.

That is, the present invention is a method for preventing a nozzle or a pipe for connecting a measuring device that monitors a process state of (meth)acrylic and esters thereof and an apparatus for handling (meth)acrylic acid and esters thereof from being clogged with a polymerisate or a solid, wherein:

the nozzle or the pipe is connected to a gas-phase part and/or a liquid-phase part of the apparatus for handling (meth)acrylic acid and esters thereof; and

the method comprises:

in the nozzle or the pipe connected to the gas-phase part of the apparatus, providing a gas blowing inlet and blowing a gas comprising at least one of an inert gas, oxygen, and a gas having an effect of inhibiting polymerization into the nozzle or the pipe from the gas blowing inlet at a flow rate of 0.03 to 1 m/second; and

in the nozzle or the pipe connected to the liquid-phase part of the apparatus, providing a liquid blowing inlet and blowing a liquid medium into the nozzle or the pipe from the liquid blowing inlet at a flow rate of 0.03 to 1 m/second.

Hereinafter, the present invention will be described in detail.

The present invention is a method for preventing a nozzle or a pipe for connecting a measuring device that monitors a process state of (meth)acrylic acid and esters thereof and an apparatus for handling the (meth)acrylic acid and esters thereof from being clogged with polymerisates or solids.

In the present invention, an “apparatus for handling (meth)acrylic acid and esters thereof” refers to an apparatus that can handle anyone of or both of (meth)acrylic acid and esters thereof.

In the present invention, a “polymerisate or solid” refers to a polymer of (meth)acrylic acid and esters thereof. Examples of the (meth)acrylic ester include an ester that is easily polymerized upon handling in reaction or distillation to yield a polymerisate, such as methyl ester, ethyl ester, n-propyl ester, isopropyl ester, n-butyl ester, isobutyl ester, 2-ethylhexyl ester, isononyl ester, n-nonyl ester, hydroxyethyl ester, ethoxyethyl ester, and dimethoxyaminoethyl ester of (meth)acrylic acid.

In the present invention, the “apparatus for handling (meth)acrylic acid and esters thereof (hereinafter, referred to as “handling apparatus”)” includes all apparatuses used in the production and storage of (meth)acrylic acid and esters thereof, such as reaction, distillation, extraction, absorption, storage, and heat exchange. Specific examples thereof include a reactor, a distillation column, a collection column, an evaporator, a heat exchanger, a tank, and an extraction tank.

The “measuring device that monitors a process state of (meth)acrylic acid and esters thereof” is for observing conditions of pressure, temperature, and liquid level, and the like, and specific examples thereof include analyzers such as a manometer, a liquid level indicator, a thermometer, an oxygen analyzer, and a pH meter. More particularly, the manometer includes a diaphragm type manometer, a bellows type manometer, and a Bourdon tube type manometer, and the like. Examples of the thermometer include a thermocouple thermometer, a resistance thermometer, and a bimetal type thermometer, and the like. Examples of the liquid level indicator include a pressure type liquid level indicator, a differential pressure type liquid level indicator, and a gauge glass, and the like.

In the present invention, a nozzle or a pipe is provided to connect the aforementioned handling apparatus and the measuring device. The nozzle or the pipe is connected to a gas-phase part and/or a liquid-phase part of the handling apparatus. The gas-phase part of the handling apparatus is a part occupied by a gas in the handling apparatus when the handling apparatus is operated. The liquid-phase part of the handling apparatus is a part occupied with a liquid in the handling apparatus when the handling apparatus is operated.

Then, in the nozzle or the pipe connected to the gas-phase part of the apparatus, a gas blowing inlet is provided, and a gas comprising an inert gas, oxygen, or a gas having the effect of inhibiting polymerization is blown in from the gas blowing inlet. In the nozzle or the pipe connected to the liquid-phase part of the apparatus, a liquid blowing inlet is provided, and a liquid medium is blown in from the liquid blowing inlet. That is, in the present invention, to the handling apparatus, the gas blowing inlet is a blowing inlet opened to the gas-phase part of the handling apparatus. To the handling apparatus, the liquid blowing inlet is a blowing inlet opened to the liquid-phase part of the handling apparatus. The blowing inlets are connected with a proper means chosen from a means for blowing a gas and a means for blowing a liquid medium.

Note that in a part that can serve either as a gas-phase part or a liquid-phase part depending on the operational conditions of the handling apparatus, a blowing inlet connected to both the means for blowing a gas and the means for blowing a liquid medium is provided to blow a proper medium chosen from the aforementioned gas and liquid medium in.

As a method for blowing the gas or liquid medium into the nozzle or the pipe, it may be performed that providing a blowing inlet 4 in a part of a nozzle 2 for connecting a handling apparatus 1 and a measuring device 3 to each other as shown in FIG. 1 and blowing a desired gas or liquid medium from it.

As described above, merely blowing a specified gas into the nozzle or the like connected to the gas-phase part has problems of, for example, an increased size of the decompressing apparatus and occurrence of polymerization in the apparatus when the process is performed under reduced pressure, while a step of treating discharged gas is required when the process is performed under atmospheric pressure. Merely blowing a specified liquid into the nozzle or the like connected to the liquid-phase part has a problem of the size of a pump that discharges the liquid must be increased, resulting in high cost.

In the present invention, to solve the aforementioned problems, the flow rate of the aforementioned gas blown in from the gas blowing inlet is set to 0.03 to 1 m/second, preferably 0.15 to 0.5 m/second. On the other hand, the flow rate of the aforementioned liquid blown into the liquid blowing inlet is set to 0.03 to 1 m/second, preferably 0.15 to 0.5 m/second.

The nozzle or the pipe is not particularly limited and includes those usually used. Specifically, a pipe having a diameter of 3/4 inch to 4 inches may be given.

When a flow rate of the gas blown in is less than 0.03 m/second or a size of the nozzle or the pipe is greater than 4 inches, generation of a polymer is liable to occur because the process fluid is not discharged sufficiently. When the flow rate is too high, cost for production or handling increases because the amount of the discharged gas from the apparatus increases.

When a flow rate of the liquid medium blown in is less than 0.03 m/second or a size of the nozzle or the pipe is greater than 4 inches, generation of a polymer is liable to occur because the process fluid is not discharged sufficiently. When the flow rate is too high, a size of the pump for blowing the liquid medium increases, and as a result, driving power such as electricity, steam, or gas becomes large, thereby cost for production or handling increases.

When the flow rate of the blowing gas or liquid medium is above 1 m/second, polymerization may occur in the handling apparatus because the flow rate of gas or liquid in the handling apparatus in which the nozzle is provided is affected and causes disturbance therein, and the values measured by the measuring device may involve errors.

At least one of an inert gas, oxygen, and a gas having the effect of inhibiting polymerization is blown into the nozzle or the like connected to the gas-phase part. Examples of the inert gas as used herein include nitrogen, argon, carbon dioxide, and steam, and the like. On the other hand, examples of the gas having the effect of inhibiting polymerization include nitrogen oxides (NOx). The gases may be used either singly or in combinations.

A rate of the inert gas and oxygen or the gas having the effect of inhibiting polymerization contained in the gas blown into the nozzle or the like connected to the gas-phase part is preferably 0/100 to 80/20, and more preferably 0/100 to 50/50. When the inert gas and oxygen or the gas having the effect of inhibiting polymerization are used in combination, the mixed ratio is preferably “inert gas:(oxygen+gas having the effect of inhibiting polymerization)=2:98 to 96:4”.

The liquid that is blown into the nozzle or the like connected to the liquid-phase part may be a simple solvent so long as flown at a specified flow rate. Examples of such the liquid include liquid media and liquids comprising a polymerization inhibitor. Among them, the liquid comprising a polymerization inhibitor is preferable as the aforementioned liquid. Examples of the polymerization inhibitor include: phenol compounds such as hydroquinone, methoxyhydroquinone (methoquinone), pyrogallol, catechol, and resorcin; N-oxyl compounds such as tertiary butyl nitrooxide, 2,2,6,6-tetramethyl-4-hydroxypiperidyl-1-oxyl, 2,2,6,6-tetramethylpiperidinooxyl, 4-hydroxy-2,2,6,6-tetramethylpiperidinooxyl, and 4,4′,4″-tris-(2,2,6,6-tetramethylpiperidinooxyl)phosphi te; phenothiazine compounds such as phenothiazine, bis-(α-methylbenzyl)phenothiazine, 3,7-dioctylphenothiazine, and bis-(α,α′-dimethylbenzyl)phenothiazine; copper-based compounds such as copper(II) chloride, copper acetate, copper carbonate, copper acrylate, copper dimethyldithiocarbamate, copper diethyldithiocarbamate, copper dibutyldithiocarbamate, and copper salicylate; manganese salt compounds such as manganese acetate; phenylenediamines such as p-phenylenediamine; nitroso compounds such as N-nitrosodiphenylamine; ureas such as urea; and thioureas such as thiourea. Those compounds may be used singly or in combination of two ore more compounds.

The polymerization inhibitor, which is liquid or solid at room temperature, can prevent polymerization of acrylic monomers when it is present in small amounts, so that the polymerization inhibitor is not used solely but is used as a solution or slurry of a predetermined liquid medium. In the present invention, these are collectively referred to as “liquids comprising a polymerization inhibitor”.

As the liquid medium, water and organic solvents are used. Examples of the organic solvents include: alcohols such as methanol, ethanol, and butyl alcohol; ketones such as acetone, methyl ethyl ketone, and methyl isobutyl ketone; carboxylic acids such as acetic acid, propionic acid, acrylic acid, and methacrylic acid; aromatic hydrocarbons such as benzene, toluene, and xylene; methyl acetate; butyl acetate; methyl acrylate; butyl acrylate; methyl methacrylate; and ethyl methacrylate, and the like. Those compounds may also be used as a mixture. Examples of the mixture that may be used include a mixture of water and toluene, a mixture of water and acrylic acid, and a crude acrylic acid comprising a dimer or trimer of acrylic acid (e.g., a column bottom liquid of an acrylic acid distillation column).

The concentration of the polymerization inhibitor in a liquid comprising the polymerization inhibitor is twice of the saturation solubility or less, preferably once of the saturation solubility or less. At or above the saturation solubility, undissolved polymerization inhibitor exists in the liquid to form a slurry liquid. However, even if this remains in the apparatus for handling (meth)acrylic acid and esters thereof, there occurs no problem since it is dissolved in a liquid in the apparatus during normal operation.

In an apparatus for producing or handling (meth)acrylic acid and esters thereof, prevention of polymerization of the substance and handling of the substance can be realized at low cost by blowing the aforementioned specified gas or liquid into the connection nozzle or pipe for connecting to a measuring device for monitoring a process state that is connected to the gas-phase part or liquid-phase part at a flow rate within the aforementioned range.

### BRIEF DESCRIPTION OF THE DRAWINGS

FIG. 1 is a diagram showing one example of provision of a blowing inlet used in the method for preventing clogging of the apparatus for handling (meth)acrylic acid and esters thereof according to the present invention.

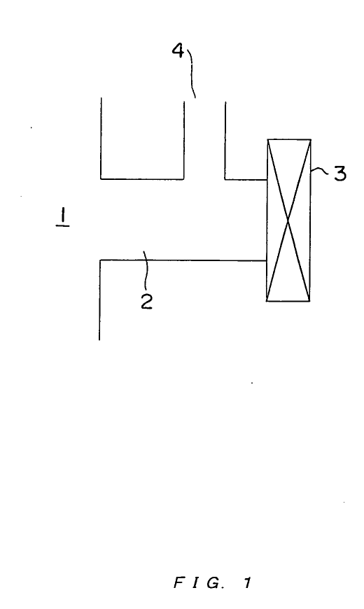

### BEST MODE FOR CARRYING OUT THE INVENTION

Hereinafter, the present invention will be described in more detail by examples and comparative examples. However, the present invention should not be limited by the following examples without departing from the gist thereof.

### EXAMPLE 1

By performing operation using a distillation column having a diameter of 1400 mm, 7.5 t/h of an acrylic acid solution containing 2.9 wt % of acetic acid and 6 wt % of toluene was distilled at a top pressure of 6 kPa and a top temperature of 50° C., to obtain acrylic acid that contained no toluene or acetic acid from the bottom. The operation was monitored by manometers installed on 3/4-inch nozzles provided in the gas-phase parts on the upper and lower parts of the distillation appliance, respectively. At the same time, air (oxygen concentration; 21 mol %) was continuously blown into the nozzle at a flow rate of 1 m/second. The operation was continued in this manner and stable operation was achieved for one month.

### EXAMPLE 2

By performing operation using the same distillation apparatus as that used in Example 1, 7 t/h of an acrylic butyl ester solution containing 0.5 wt % n-butanol and 1 wt % of water was distilled at a top pressure of 6 kPa and a top temperature of 52° C., and acrylic butyl ester that contained no n-butanol and water was obtained from the bottom. A liquid level indicator was provided on the lower part of the distillation appliance by using a 3-inch nozzle and the operation was monitored. At the same time, a gas comprising oxygen (concentration of oxygen 6 mol %, nitrogen 94 mol %) was continuously blown into a low pressure side nozzle (gas-phase part) of the liquid level indicator at a flow rate of 0.03 m/second. On the other hand, a liquid having dissolved 3 wt % of hydroquinone in acrylic butyl ester was continuously blown into the high pressure side nozzle (liquid-phase part) of the liquid level indicator at a flow rate of 0.3 m/second. Operation was continued in this manner and stable operation was achieved for one month.

### EXAMPLE 3

By performing operation using a high-boiling material decomposition apparatus, 500 kg/h of a solution of 30 wt % of acrylic acid containing 0.03 wt % of copper acrylate and 65 wt % of acrylic acid dimer was distilled at a top pressure of 70 kPa and a top temperature of 143° C., and acrylic acid that contained no acrylic acid dimer was obtained from the top. A liquid level indicator was provided on the lower part of the distillation appliance by using a 3-inch nozzle and the operation was monitored. At the same time, nitrogen was continuously blown into a low pressure side nozzle (gas-phase part) of the liquid level indicator at a flow rate of 0.06 m/second. On the other hand, a column bottom liquid of the high-boiling material decomposition apparatus containing 0.1 wt % of copper acrylate as a polymerization inhibitor was continuously blown into the high pressure side nozzle (liquid-phase part) of the liquid level indicator at a flow rate of 0.03 m/second. The operation was continued in this manner and stable operation was achieved for one month.

### COMPARATIVE EXAMPLE 1

By performing operation using the distillation apparatus of Example 1, 7.5 t/h of an acrylic acid solution containing 2.9 wt % of acetic acid and 6 wt % of toluene was distilled at a top pressure of 6 kPa and a top temperature of 50° C., and the operation was monitored by manometers installed on 3/4-inch nozzles provided on the upper and lower parts of the column, respectively. After 1 week from the start of the operation, the pressure at the bottom increased gradually and hence the operation was stopped after 2 weeks from the start of the operation and the apparatus was checked. It was confirmed that the entire nozzle was clogged with a polymer of acrylic acid.

### COMPARATIVE EXAMPLE 2

By performing operation using the decomposition apparatus of Example 3, 500 kg/h of a solution of 30 wt % of acrylic acid and 70 wt % of acrylic acid dimer was decomposed at a top pressure of 70 kPa and a top temperature of 143° C. and the operation was monitored by a liquid level indicator installed using a 3-inch nozzle on the lower part of the distillation apparatus. After 2 days from the start of the operation, liquid level indication on the bottom became unstable and hence the operation was stopped after 1 week from the start of the operation and the apparatus was checked. It was confirmed that the high pressure side nozzle of the liquid level indicator was partly clogged with a polymer of acrylic acid and the low pressure side nozzle of the liquid level indicator was entirely clogged with the polymer of acrylic acid.

### INDUSTRIAL APPLICABILITY

According to the present invention, a nozzle or a pipe for connecting a measuring device that monitors a process state of (meth)acrylic acid and esters thereof and an apparatus for handling (meth)acrylic acid and esters thereof to each other can be efficiently prevented from being clogged with polymerisates or solids, so that the apparatus for handling (meth)acrylic acid and the like can be operated stably and efficiently to thereby decrease cost for production or storage of (meth)acrylic acid.

1. A method for preventing a nozzle or a pipe for connecting a measuring device that monitors a process state of (meth)acrylic acid and esters thereof and an apparatus for handling the (meth)acrylic acid and esters thereof from being clogged with a polymerisate or a solid, wherein:
the nozzle or the pipe is connected to a gas-phase part and/or a liquid-phase part of the apparatus for handling (meth)acrylic acid and esters thereof; and
the method comprises:
in the nozzle or the pipe connected to the gas-phase part of the apparatus, providing a gas blowing inlet and blowing a gas comprising at least one of an inert gas, oxygen, and a gas having an effect of inhibiting polymerization into the nozzle or the pipe from the gas blowing inlet at a flow rate of 0.03 to 1 m/second; and
in the nozzle or the pipe connected to the liquid-phase part of the apparatus, providing a liquid blowing inlet and blowing a liquid medium into the nozzle or the pipe from the liquid blowing inlet at a flow rate of 0.03 to 1 m/second.

2. The method according to claim 1, wherein the method comprises blowing a liquid comprising a polymerization inhibitor as the liquid medium from the liquid blowing inlet.
